(19) United States
(12) Patent Application Publication
(10) Pub. No.: US 2026/0182681 A1
(43) Pub. Date: Jul. 2, 2026

(54) FACE SHIELD ASSEMBLY

(71) Applicant: APJ Health Ltd, Lancashire (GB)

(72) Inventors: Alan Graeme KER, Lancashire (GB); James SEAL, Lancashire (GB)

(73) Assignee: APJ Health Ltd, Lancashire (GB)

(21) Appl. No.: 19/008,301

(22) Filed: Jan. 2, 2025

(51) Int. Cl. A41D 13/11

(52) U.S. Cl. CPC A41D 13/1161; A61F 9/04; A61F 9/045

(57) ABSTRACT

A face shield assembly (1) comprises a brow band (2) having an inner flexible flange portion (21) configured to contact the brow of a wearer, a transparent face guard (3) releasably attachable to the brow band and configured to provide a barrier to a face of a wearer and an elongate securing strap (4) releasably attachable to the brow band for securing the face shield to the head of a wearer. The brow band (2) also has an outer mounting portion (22) to which the face guard (3) is releasably attachable and a spacer element (23) connecting the inner flexible flange portion (21) and outer mounting portion (22) such that they are spaced from one another. The inner flexible flange portion (21), outer mounting portion (22) and spacer element (23) define an upwardly open channel section (20).

This invention relates generally to face shield assemblies, and more particularly to head mountable face guards. More specifically, although not exclusively, this invention relates to head mountable face shield assemblies that are reusable and/or recyclable.

The use of personal protective equipment (“PPE”), and in particular face shields, has increased enormously in recent times as a result of the Covid-19 pandemic, particularly by clinicians in the healthcare sector, and it seems highly likely that the widespread use of face shields will continue in the healthcare sector for many years to come.

The Applicants have observed that the vast majority of face shields are designed to be single-use which must be disposed of safely and hygienically after use. The single-use nature of such face shields requires large amounts of raw materials and energy in their production, and the nature of the products makes it more difficult to reuse or recycle them safely, with the result that most single-use face shields are disposed of as clinical waste or in landfill.

It has also been observed that reusable PPE face shields almost universally use a foamed rubber headband to engage the forehead of a wearer and an elastic band passing around the rear of a wearer's head, both of which collect pathogens and which are difficult to clean and sterilise effectively. The existing reusable PPE face shields are also generally assembled using adhesives, which can degrade and discolour during storage (which can be for months, or even years) and which also makes recycling difficult.

It is believed that there is a clinical and commercial desire for a reusable face shield for use as PPE which can be quickly and effectively cleaned and sterilised for reuse, unlike the known reusable face shields.

It is therefore a first non-exclusive object of the invention to provide an improved face shield that overcomes, or at least mitigates, the aforementioned drawbacks of the prior art.

Accordingly, a first aspect of the invention provides a face shield assembly comprising: a brow band comprising an inner flexible flange portion configured to contact the brow of a wearer; a face guard releasably attachable to the brow band and configured to provide a barrier to a face of a wearer; and an elongate securing strap releasably attachable to the brow band for securing the face shield to the head of a wearer.

It will be appreciate that this arrangement provides a face shield which can dismantled into its constituent components to allow for the cleaning or replacement of components or allow components to be recycled at the end of their useful life.

The brow band may further comprise an outer mounting portion, e.g. to which the face guard is releasably attachable. The brow band may also include a spacer element connecting the inner flexible flange portion and outer mounting portion such that they are spaced from one another.

The inner flexible flange portion, outer mounting portion and spacer element may define a channel section, e.g. an upwardly open channel section.

The channel section may comprise a Z profile or a U profile.

In some examples, the channel section is resiliently deformable. The spacer element may be provided by a web section and may connect the inner flexible flange portion and outer mounting portion. The channel section may be flexible.

The outer mounting portion may comprise an outer flexible flange portion.

The inner flexible flange portion may be movable relative to the outer flexible flange portion.

The outer flexible flange portion may define a constant spacing from the inner flexible flange portion. The constant spacing may be provided by the spacer element and/or web portion. The inner flexible flange portion and/or outer flexible flange portion may substantially planar in vertical cross-section. The inner flexible flange portion may describe an inner wall. The outer flexible flange portion may describe an outer wall.

The inner flexible flange portion may have an upper free end or a lower free end. The outer flexible flange portion may have an upper free end or a lower free end.

The brow band may comprise a respective tab extending from each end of the outer flexible flange portion. Each tab may have a means for attaching the face guard and/or elongate securing strap to the brow band.

Each tab may be an extension of the inner flexible flange portion or the outer flexible flange portion and/or be formed integrally therewith.

Each tab may comprise one or more cleats or projections for attaching the face guard and/or securing strap to the brow band.

At least one cleat or projection may comprise a neck and head portion. The neck may connect the tab and head portion.

Each tab may comprise an elongate slot. The elongate slot may be configured to receive an edge portion of the face guard, e.g. to prevent rotation thereof relative to the brow band.

The brow band may be arcuate.

The brow band, inner flexible flange portion, outer flexible flange portion and/or spacer element may be non-absorbent.

The inner flexible flange portion and/or outer mounting portion may be curved or arcuate. The brow band may be unitary. The brow band may be of an injected moulded single piece construction.

The brow band may be formed of a plastics or polymeric material.

The face guard may comprise one or more apertures corresponding to the one or more cleats or projections. The one or more apertures may be configured to receive the projections so as to releasably attach the face guard to the brow band.

At least part of the face guard may be configured to be retained on the brow band between the brow band and securing strap.

The securing strap may comprise a pair of apertures connected by a neck. A first of the apertures may be larger, e.g. in diameter, than the head portion. A second of the apertures may be smaller, e.g. in diameter, than the head portion. The neck of the at least one cleat or projection may be configured to be received within the second of the apertures, e.g. via the first of the apertures, in a snap-fit type arrangement.

The securing strap may be adjustable in length.

The securing strap may have a cable portion having a plurality of teeth and a releasable pawl configured to engage the teeth.

The securing strap may have a stretchable portion. The securing strap or stretchable portion may comprise a lattice structure formed of a strip of plastics or polymeric material having a plurality of cut-outs or apertures.

The securing strap may have a cable portion having a plurality of teeth along a part of its length and a stretchable portion along another part of its length.

In some examples, the securing strap is unitary.

Each of the face guard and securing strap may be removable from the brow band.

The face guard may be formed of a substantially transparent plastics or polymeric material. The face guard may be formed of a sheet of material.

According to a second aspect of the invention, there is provided a brow band for use in a face shield assembly, the brow band comprising: an inner flexible flange portion configured to contact the brow of a wearer, an outer flexible flange portion comprising attachment means for releasable attachment of a face guard and a spacer element connecting the inner flexible flange portion and outer flexible flange portion.

The inner flexible flange portion, outer flexible flange portion and spacer element may define a resiliently deformable channel section. The channel section may comprise a Z profile or U profile. The channel section may have a respective tab extending from each end thereof. Each tab may have a means for releasably attaching a face guard and/or elongate securing strap to the brow band.

According to a third aspect of the invention, there is provided a head mountable face shield assembly comprising a brow band as described above, a face guard as described above and a securing strap as described above.

For the avoidance of doubt, any of the features described herein apply equally to any aspect of the invention. For example, any features of the face shield assembly relevant or applicable to the brow band may be applied thereto and vice versa.

Another aspect of the invention provides a computer program element comprising and/or describing and/or defining a three-dimensional design for use with a simulation means or a three-dimensional additive or subtractive manufacturing means or device, e.g. a three-dimensional printer or CNC machine, the three-dimensional design comprising an embodiment of the face shield assembly, brow band, face guard and/or securing strap described above.

A yet further aspect of the invention provides the computer program element embodied on a computer readable medium.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the invention, whether or not these are expressly claimed. The applicant reserves the right to change any originally filed claim or file any new claim accordingly, including the right to amend any originally filed claim to depend from and/or incorporate any feature of any other claim although not originally claimed in that manner.

Embodiments of the invention will now be described by way of example only with reference to the accompanying drawings in which:

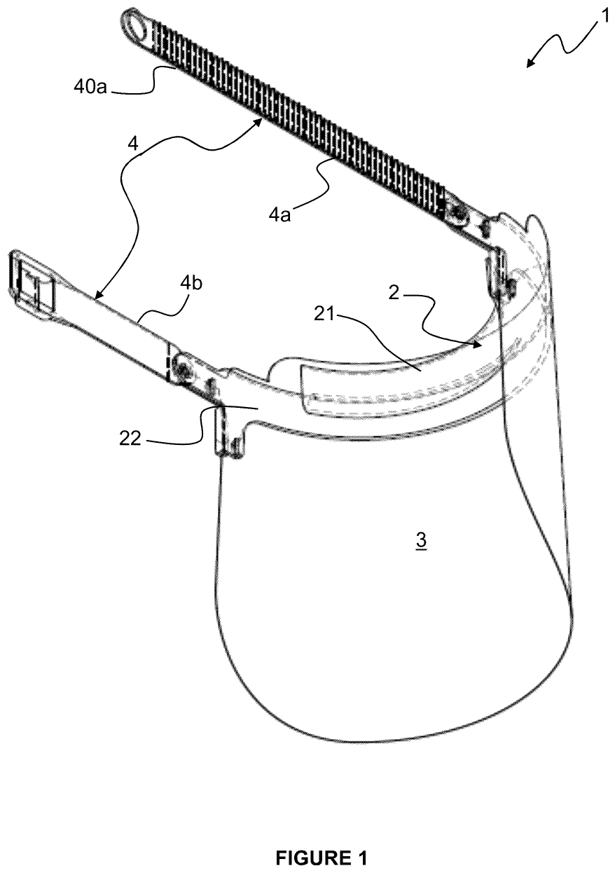

FIG. 1 is a front perspective view of an embodiment of face shield assembly in accordance with the invention;

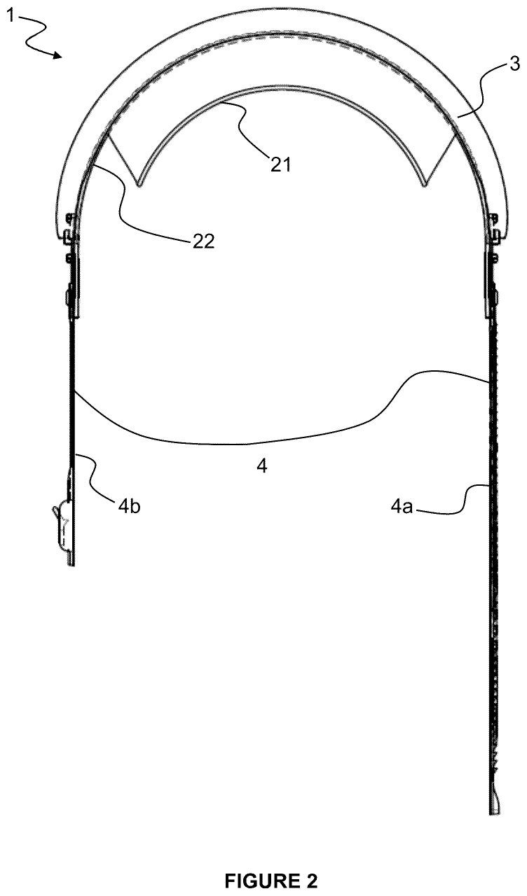

FIG. 2 is a plan view of the face shield assembly of FIG. 1;

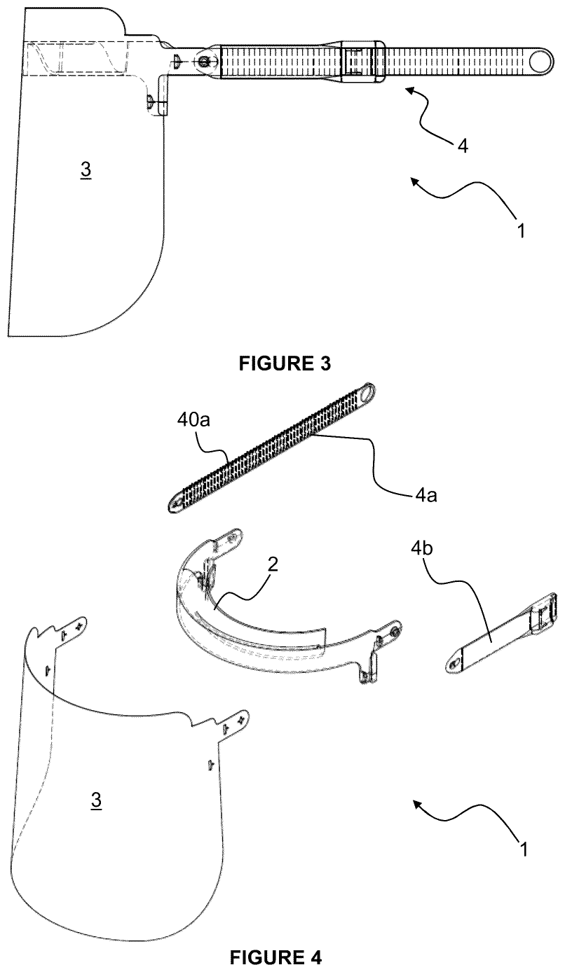

FIG. 3 is a side view of the face shield assembly of FIG. 1;

FIG. 4 is a front perspective exploded view of the face shield assembly of FIG. 1;

FIG. 5 is a exploded plan view of the face shield assembly of FIG. 1;

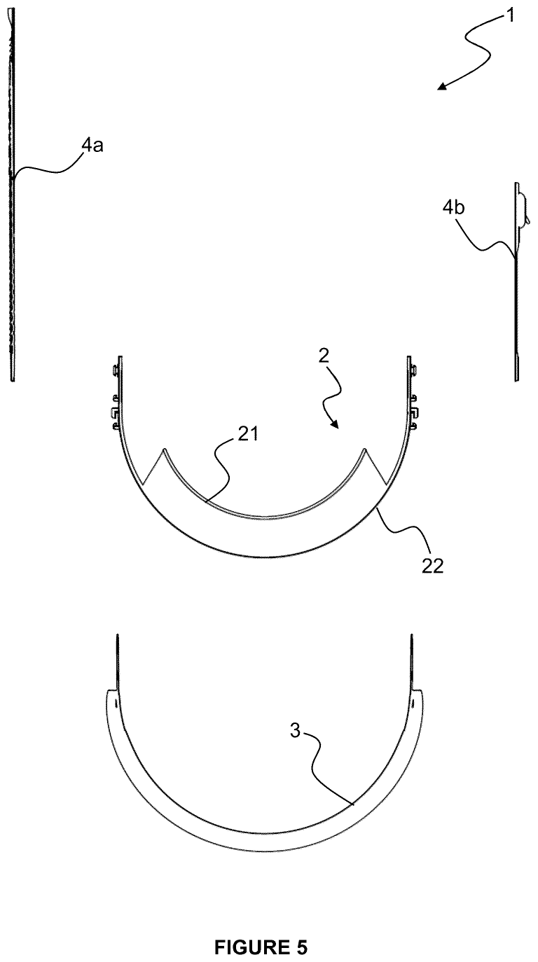

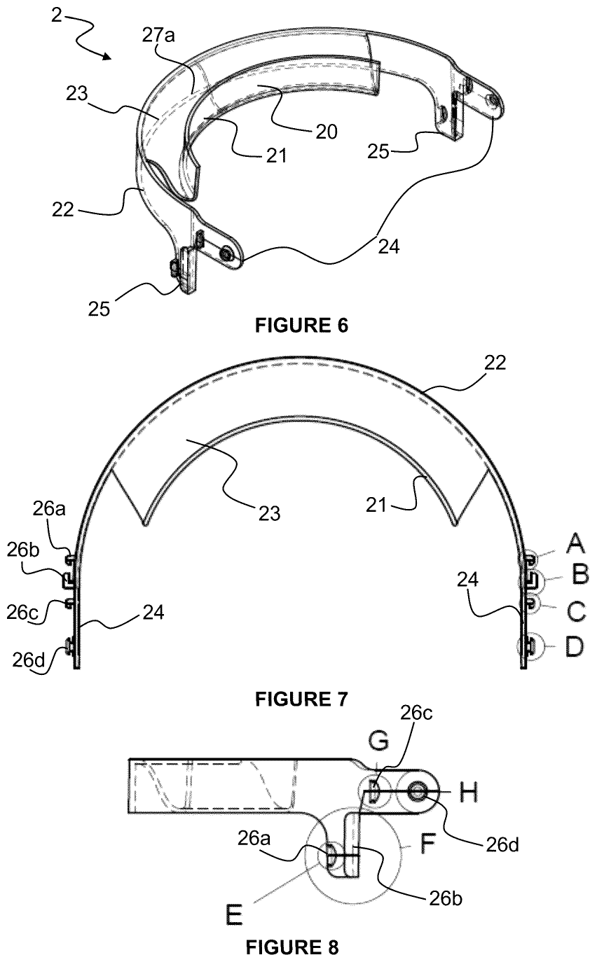

FIG. 6 is a rear perspective view of a brow band which forms part of the face shield assembly of FIG. 1;

FIG. 7 is a plan view of the brow band of FIG. 6;

FIG. 8 is a side view of the brow band of FIGS. 6 and 7;

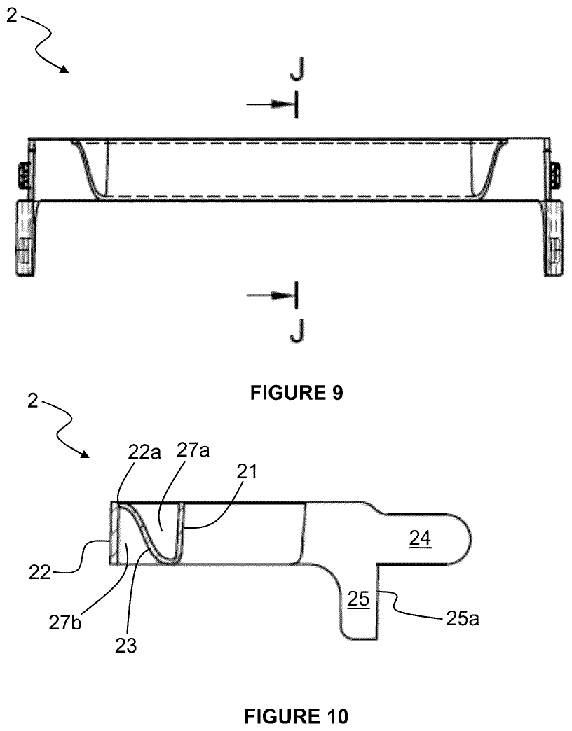

FIG. 9 is a rear view of the brow band of FIGS. 6 to 8;

FIG. 10 is a sectional view through plane J-J of FIG. 9;

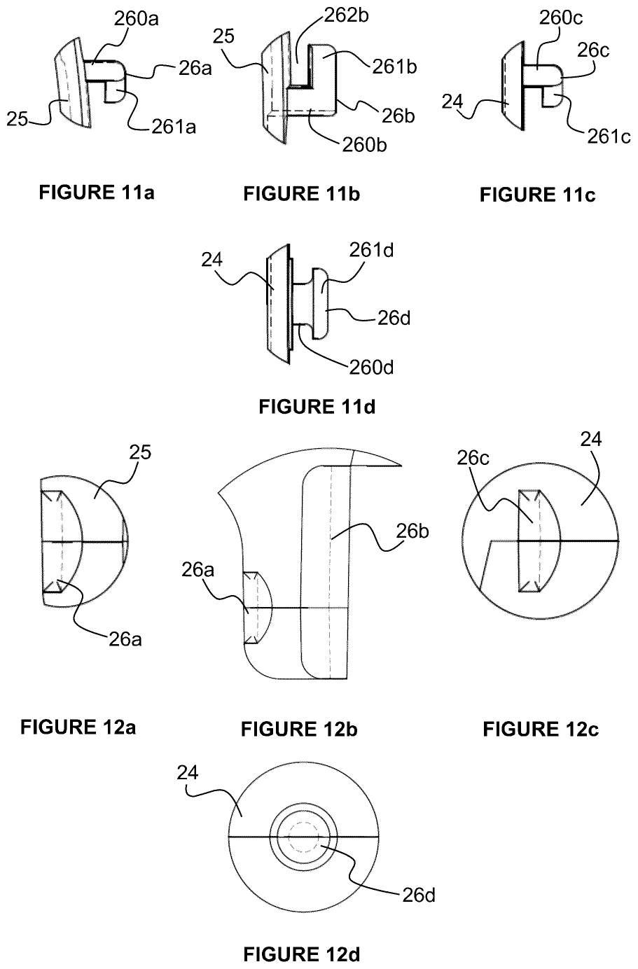

FIG. 11a is a detail view of region A of FIG. 7;

FIG. 11b is a detail view of region B of FIG. 7;

FIG. 11c is a detail view of region C of FIG. 7;

FIG. 11d is a detail view of region D of FIG. 7;

FIG. 12a is a detail view of region E of FIG. 8;

FIG. 12b is a detail view of region F of FIG. 8;

FIG. 12c is a detail view of region G of FIG. 8;

FIG. 12d is a detail view of region H of FIG. 8;

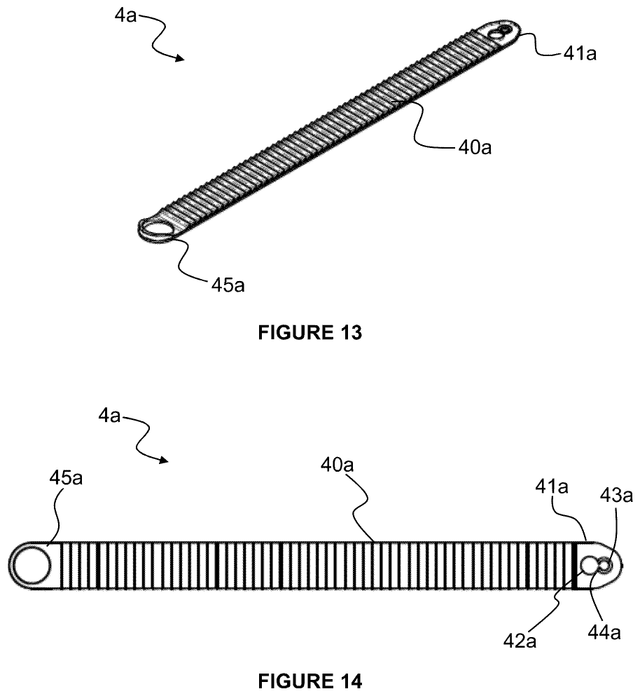

FIG. 13 is a perspective view of a cable portion of a securing strap which forms part of the face shield assembly of FIG. 1;

FIG. 14 is a plan view of the cable portion of FIG. 13;

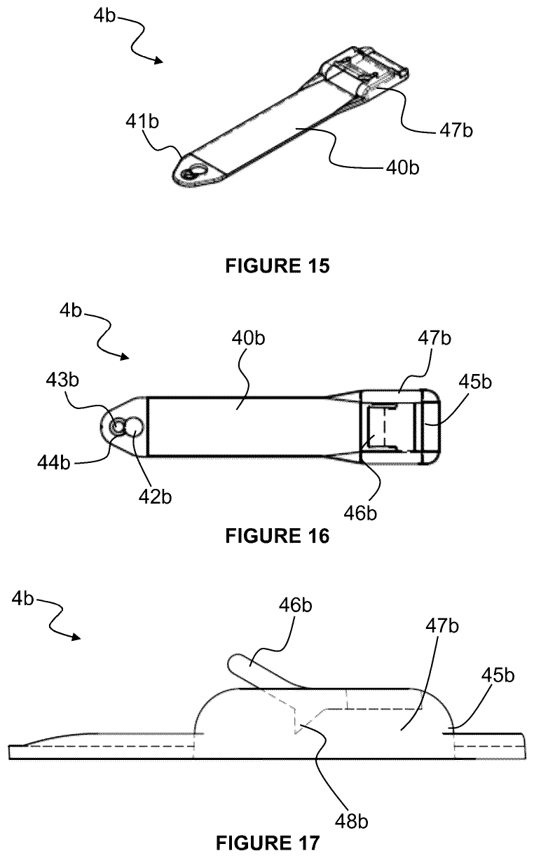

FIG. 15 is a perspective view of the releasable pawl of the securing strap which forms part of the face shield assembly of FIG. 1;

FIG. 16 is a plan view of the releasable pawl of FIG. 15;

FIG. 17 is a side view of the releasable pawl of FIG. 15;

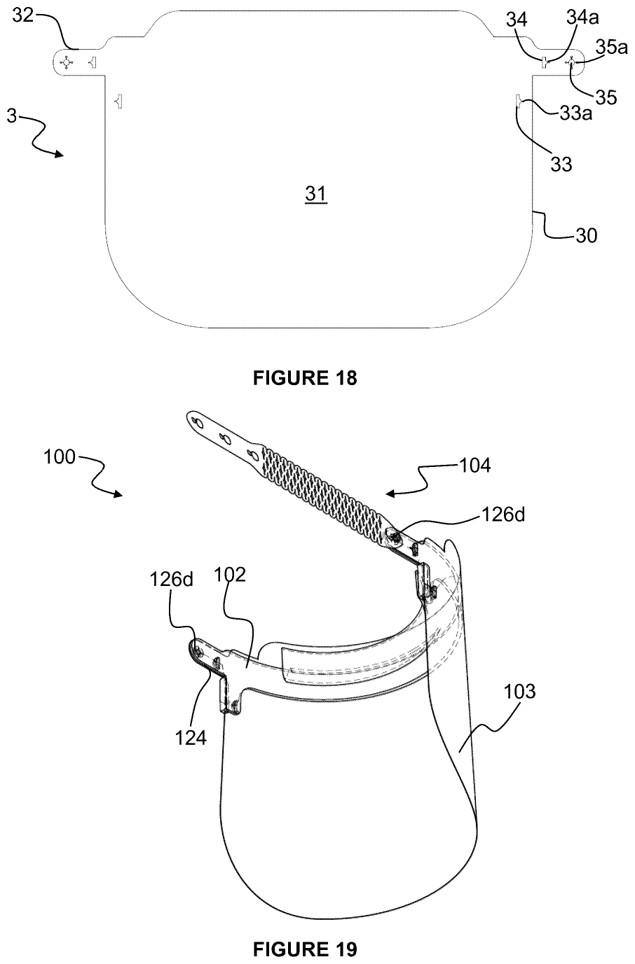

FIG. 18 is a front view of the face guard which forms part of the face shield assembly of FIG. 1 in an uncurved state, prior to being secured to the brow band;

FIG. 19 is a front perspective view of a second embodiment of face shield assembly in accordance with the invention;

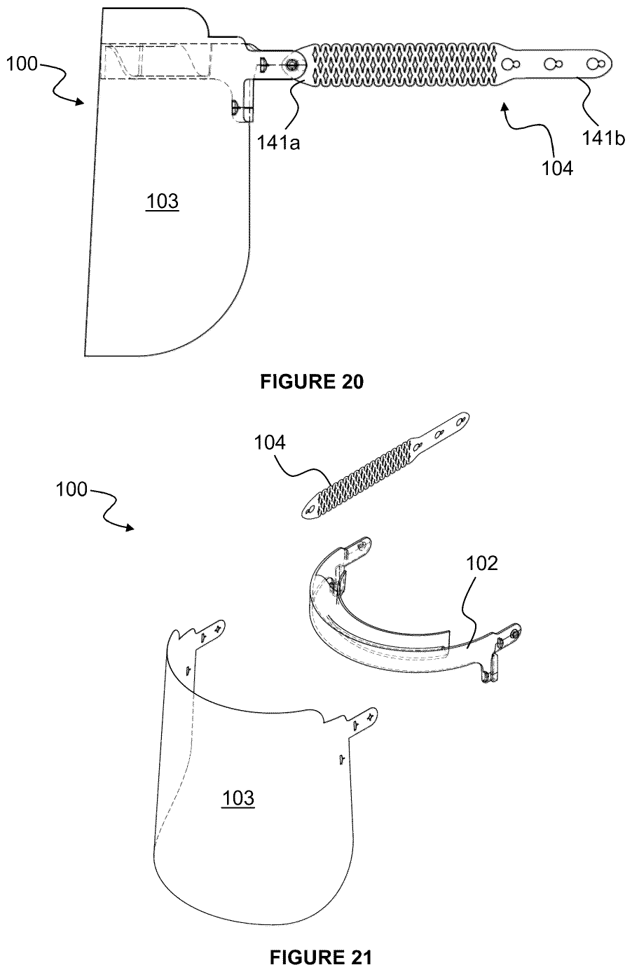

FIG. 20 is a side view of the face shield assembly of FIG. 19;

FIG. 21 is a front perspective exploded view of the face shield assembly of FIG. 19;

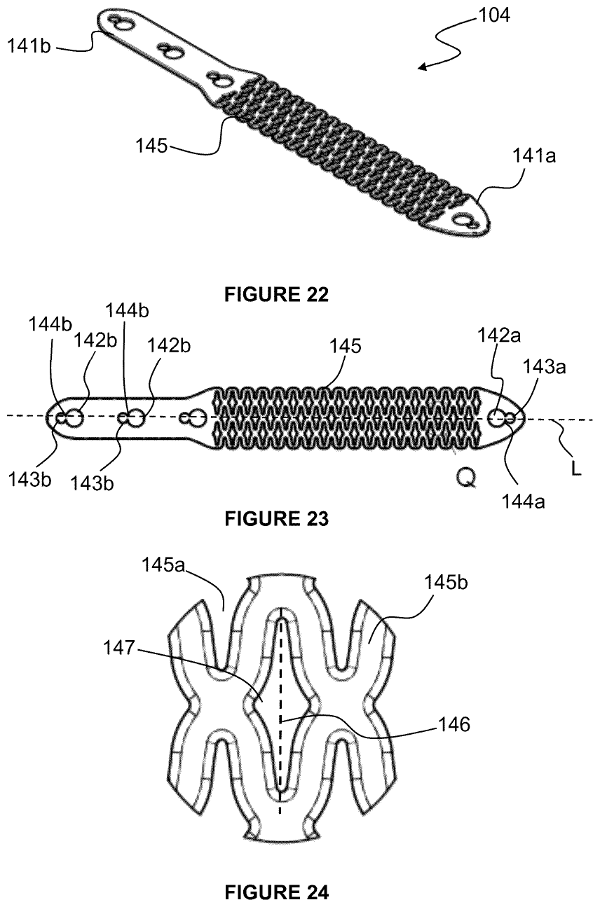

FIG. 22 is a perspective view of the securing strap of FIG. 19;

FIG. 23 is a plan view of the securing strap of FIG. 22;

FIG. 24 is a detail view of the stretchable portion of the securing strap of FIG. 22; and

Referring now to FIGS. 1 to 5, there is shown a first embodiment of face shield assembly in accordance with the invention. The face shield assembly 1 is mountable or wearable on the head of a user and includes an arcuate brow band 2 which in use contacts the brow of a wearer and a transparent face guard 3 attached thereto. The transparent face guard 3 is removably attached to the brow band 2 and is configured to provide a barrier to a face of a wearer in order to prevent or reduce facial contact with aerosol droplets. In the present example, and as described in greater detail below, the face guard 3 is retained on the brow band 2 by being captivated between the brow band 2 and a removable securing strap 4.

The securing strap 4 is configured to secure the face shield 1 to the head of a wearer and maintain contact between the brow band 2 and the brow of a wearer. The securing strap 4 is formed from flexible plastics material, is adjustable in length and is formed of two parts in the present example; a cable portion 4a having a plurality of teeth 40a located along its length and a releasable pawl 4b configured to engage the teeth 40a. The securing strap 4 is passed around the back of the head of a wearer, in use.

Referring now to FIGS. 6 to 10, there is shown the brow band 2 in greater detail. The brow band 2 includes a curved portion having a resiliently deformable, flexible channel section 20.

The channel section 20 is a Z-profile in the present example, as shown in the cross-sectional view of FIG. 10 in particular. The channel section 20 is defined by an upstanding flexible inner wall 21 arranged to contact the brow of a wearer and an outer upstanding flexible wall 22 substantially parallel to, and spaced from, the inner wall 21. The inner wall 21 is shorter than the outer wall 22 and a flexible web section 23 extending between the lower edge of the inner wall 21 and the upper edge of the outer wall 22 acts as a spacer element that connects the inner wall 21 and outer wall 22. An upwardly open channel 27a is defined between the inner wall 21 and the web 23 and a downwardly open channel 27b is defined between the outer wall 22 and the web 23. The inner wall 21 thereby forms a curved flexible flange which extends upwardly from the lower end of the web 23 and the outer wall 22 thereby forms a curved flexible flange which extends downwardly from the upper end of the web 23. Relative movement between the inner wall 21 and the outer wall 22 is provided by virtue of flexure of the connection between the web section 23 and each of the inner wall 21 and outer wall 22. In the present example, the inner wall 21, the outer wall 22 and web section 23 are unitary and are formed as a one-piece moulding.

Extending from each end of the channel section 20 is a respective rearwardly extending elongate tab 24 having means for attaching the face guard 3 and securing strap 4 to the brow band 2. In the present example, the outer wall 22 is formed as a strip of polymeric or plastics material and the tabs 24 are provided as an extension thereof, and are formed integrally therewith. Each tab 24 also includes an elongate extension part 25 extending substantially orthogonally downwardly therefrom and having further means for attaching the face guard 3.

In the present example, the means for attaching the face guard 3 and securing strap 4 to the brow band 2 includes sets of projections 26a, 26b, 26c and 26d. The projections 26a; 26d are provided in pairs, a respective one of the projections 26a; 26d located on the outer face of each of the tabs 24/extension parts 25.

A first projection 26a is shown in region A of FIG. 7, region E of FIG. 8 and FIGS. 11a and 12a. A second projection 26b is shown in region B of FIG. 7, region F of FIG. 8 and FIGS. 11b and 12b. A third projection 26c is shown in region C of FIG. 7, region G of FIG. 8 and FIGS. 11c and 12c. A fourth projection 26d is shown in region D of FIG. 7, region H of FIG. 8 and FIGS. 11d and 12d.

The first projection 26a is located on the extension part 25 and has an outwardly projecting arm 260a extending substantially perpendicularly therefrom. A return portion 261a extends substantially orthogonally from the end of the arm 260a and substantially parallel to the extension part 25 and towards the second projection 26b to a free end.

The second projection 26b is also located on the extension part 25 and has an elongate arm 260b extending substantially perpendicularly from the rearmost elongate edge 25a of the extension part 25 for the whole length of the edge 25a. The elongate arm 260b extends along the extension part 25 a greater distance than the arm 260a. A return portion 261b extends substantially orthogonally from the end of the arm 260b, substantially parallel to the extension part 25 and towards the first projection 26a to a free end, defining a receiving channel 262b for an edge of the face guard 3.

In use, the first projection 26a and second projection 26b work together to secure a portion of the face guard 3 on the brow band 2. As described in greater detail below, an outer edge 30 (FIG. 18) of the face guard 3 is configured to be received within the channel 262b defined by the second projection 26b in order to prevent rotation of the face guard 3 relative to the brow band 2.

The third projection 26c is located on the tab 24 and is very similar in profile to the first projection 26a. The third projection 26c has an arm 260c extending substantially perpendicularly from the tab 24. A return portion 261c extends substantially orthogonally from the end of the arm 260c, substantially parallel to the tab 24 and towards the fourth projection 26d to a free end.

The fourth projection 26d is also located on the tab 24 and is in the form of a cleat having a neck portion 260d extending from the tab 24 and an enlarged head portion 261d located at an end of the neck portion 260d.

In use, the fourth projection 26d is configured to work together with the securing strap 4 to attach a portion of the face guard 3 to the brow band 2. The fourth projection 26d is configured to firstly be received within a third aperture 35 (FIG. 18) of the face guard 3, and then secondly to be received within an aperture of the securing strap 4 (described in greater detail below) so as to captivate a portion of the face guard 3 between the tab 24 and securing strap 4.

The cable portion 4a of the securing strap 4 is shown in more detail in FIGS. 13 and 14. The cable portion 4a has a plurality of teeth 40a located along its length. At a first end of the cable portion 4a is an attachment tab 41a for attaching the cable portion 4a to the brow band 2. The attachment tab 41a includes a pair of connected apertures 42a, 43a of different sizes having a neck 44a therebetween and is configured to attach the cable portion 4a to the brow band 2 via the fourth projection 26d. A first of the apertures 42a is larger than the head portion 261d of the fourth projection 26a and a second of the apertures 43a is smaller than the head portion 261d. In order to attach the cable portion 4a to the brow band 2, the head portion 261d is received within the first aperture 42a and the neck 260d of the fourth projection 26d is then passed through the neck 44a and captivated within the second aperture 43a in a snap-fit type arrangement.

A second end of the cable portion 4a includes a pull ring 45a to allow for tightening of the securing strap 4 when the cable portion 4a is received within the releasable pawl 4b.

The releasable pawl 4b of securing strap 4 is shown in more detail in FIGS. 15 to 17. The releasable pawl 4b includes a strap portion 40b having an attachment tab 41b at one end. In a similar manner to the cable portion 4a, the attachment tab 41b includes a pair of connected apertures 42b, 43b of different sizes having a neck 44b therebetween and is configured to attach the releasable pawl 4b to the brow band 2 via the fourth projection 26d. A first of the apertures 42b is larger than the head portion 261d of the fourth projection 26a and a second of the apertures 43b is smaller than the head portion 261d. The releasable pawl 4b is attached to the brow band 2 in a similar manner to the cable portion 4a described above.

The releasable pawl 4b also includes an opening 45b leading to a latch 46b mounted to a buckle 47b. The latch 46b is allowed to move relative to the buckle 47b. The latch 46b has a pawl element 48b configured to engage the teeth 40a of the cable portion 4a.

In use, the cable portion 4a is attached to one tab 24 via a fourth projection 26d and the releasable pawl 4b is attached to the other tab 24 via the other fourth projection 26d. An end of the cable portion 4a is passed through the opening 45b of the releasable pawl 4b and pulled through such that the pawl element 48b engages the teeth 40a. To tighten the securing strap 4 the pull ring 45a is pulled towards the attachment tab 41b. The pawl element 48b prevents inadvertent loosening of the securing strap 4. In order to loosen the securing strap 4, the latch 46b is lifted so as to bring the pawl element 48b out of engagement with the teeth 40a such that the cable portion 4a can be released.

The face guard 3 is shown in more detail in FIG. 18, in an uncurved state prior to its attachment to the brow band 2. The face guard 3 is formed of a substantially transparent flexible polymeric or plastics material in this example having an outer edge 30. The face guard 3 also includes a shield portion 31 configured to provide a barrier to the face of a wearer of the face shield assembly 1. Extending from the shield portion 31 is a pair of opposed mounting tabs 32 for attaching the face guard 3 to the brow band 2.

The face guard 3 also includes a pair of first apertures 33 located on the shield portion 31 proximate the outer edge 30. Further, there is provided a pair of second apertures 34, with a respective aperture 34 located on each of the mounting tabs 32. The first apertures 33 and second apertures 34 are similar in geometry and are provided as an elongate opening. Extending from one side of the elongate opening is a slit 33a, 34a to allow the size of the opening provided by the apertures 33, 34 to be expanded.

A pair of third apertures 35 is also provided, with a respective aperture 35 located on each of the mounting tabs 32. The third apertures 35 are provided with a circular geometry and have four slits 35a rotationally spaced from one another by 90 degrees. The slits 35a allow the size of the opening provided by the third apertures 35 to be expanded. In their unexpanded state, the third apertures 35 have a diameter smaller than the head portion 261d of the fourth projection 26d. However, the slits 35a allow the third apertures 35 to expand such that the head portion 261d can be passed through.

Referring now to FIGS. 6 to 8 and FIG. 18, in order to assemble the face shield assembly 1 the face guard 3 is removably attached to the brow band 2. The face guard 3 is deformed into the arcuate shape shown in FIG. 1 and the upper part of the outer edge 30 is inserted into the elongate slot 262b provided by the second projection 26b. The first projection 26a is inserted into the first aperture 33 and the return portion 261a retains the first projection 26a therewithin. Further, the third projection 26c is inserted into the second aperture 34 in a similar manner to the first projection 26a described above. Finally, the fourth projection 26d is inserted into the third aperture 35.

Referring now to FIGS. 13 to 17, the next step in the assembly of the face shield assembly 1 is to removably attach the securing strap 4 to the brow band 2. The cable portion 4a and releasable pawl 4b are each attached to the brow band 2 via the fourth projections 26d, as described above. When the securing strap 4 is attached, a portion of the face guard 3 is captivated between the brow band 2 and each of the cable portion 4a and releasable pawl 4b.

In use, the assembled face shield assembly 1 is fitted to the head of a wearer by passing the securing strap 4 around the back of the wearer's head with the inner wall 21 of the brow band 2 in contact with the wearer's brow. The securing strap 4 is then tightened by pulling the cable portion 4a through the releasable pawl 4b until the strap is comfortably tight against the back of the wearer's head, the inner wall 21 of the brow band 2 is comfortably tight against the wearer's brow and the assembly 1 is securely retained on the wearer's head. The flexible flange portion, formed by the flexible inner wall 21, in contact with the wearer's head deforms on tightening the securing strap 4 to form a comfortable seal with the wearer's brow, which prevents the passage of airborne particles. The stretchable nature of the securing strap also helps to achieve a comfortable fit for the wearer. The face guard 3 is also effective in disrupting and reducing the flow of airborne particles towards the wearer's face and mouth.

The face shield assembly 1 can be dismantled into its constituent components, i.e. the brow guard 2, face guard 3 and securing strap 4. Each of the components is non-absorbent and can be cleaned and reused if desired. Alternatively, one or more of the components can be replaced if necessary or desirable, without requiring the replacement of the other components.

Referring now to FIGS. 19 to 24, there is shown a second embodiment of face shield 100 according to the invention, wherein like features in the first embodiment are denoted by like references incremented by ‘100’.

The face shield assembly 100 differs from face shield assembly 1 in the arrangement of the securing strap 104. In the present example, the securing strap 104 is stretchable and is provided as a unitary component. In contrast, the securing strap 4 is provided in two parts, the cable portion 4a and the releasable pawl 4b. The securing strap 104 has a first attachment tab 141a at a first end and a second attachment tab 141b at a second end.

The first attachment tab 141a includes a pair of connected apertures 142a, 143a of different sizes having a neck 144a therebetween and is configured to attach the first end of the securing strap 104 to the brow band 102 via the fourth projection 126d. The second attachment tab 141b includes three spaced pairs of connected apertures 142b, 143b to allow for adjustment of the tightness of fit of the face shield 100. The pairs of apertures 142b, 143b include apertures of different sizes having a neck 144b therebetween and are configured to attach the second end of the securing strap 104 to the brow band 102 via the other fourth projection 126d.

The securing strap 104 further includes a stretchable portion 145 shown in an unstretched condition in FIGS. 19 to 24. The stretchable portion 145 has a lattice structure described by a plurality of substantially rhomboid shaped or super-elliptical apertures 145a. The major axis 146 of each of the apertures 145a extends substantially orthogonal to a longitudinal axis L (FIG. 23) of the securing strap 104. The minor axis 147 of each of the apertures 145a extends substantially parallel to the longitudinal axis L.

The plurality of apertures 145a define a lattice or mesh of interconnected webs 145b. In use, a tensile force applied the stretchable portion 145 causes the apertures 145a to be deformed such that they are increased in the direction of the minor axis 147. This results in the overall length of the stretchable portion 145 to be increased allowing for adjustment of the face shield 100.

The second embodiment of face shield assembly 100 can be assembled, fitted to a wearer, dismantled, cleaned and reused, or one or more components can be replaced, in the same manner as the first embodiment.

It will be appreciated by those skilled in the art that several variations to the aforementioned embodiments are envisaged without departing from the scope of the invention. For example, whilst the brow band 2, 102 is described as having a channel section with a Z-profile, this need not be the case. Instead, the brow band may have a channel section having a U-profile, or any other suitable cross-sectional profile.

The invention is not restricted to the details of the foregoing embodiments.

It will also be appreciated by those skilled in the art that any number of combinations of the aforementioned features and/or those shown in the appended drawings provide clear advantages over the prior art and are therefore within the scope of the invention described herein.

1. A face shield assembly comprising:
a brow band comprising an inner flexible flange portion configured to contact the brow of a wearer;
a face guard releasably attachable to the brow band and configured to provide a barrier to a face of a wearer; and
an elongate securing strap releasably attachable to the brow band for securing the face shield to the head of a wearer.

2. A face shield assembly according to claim 1, wherein the brow band further comprises an outer mounting portion to which the face guard is releasably attachable and a spacer element connecting the inner flexible flange portion and outer mounting portion such that they are spaced from one another.

3. A face shield assembly according to claim 2, wherein the inner flexible flange portion, outer mounting portion and spacer element define an upwardly open channel section.

4. A face shield assembly according to claim 3, wherein the channel section comprises a Z profile or a U profile.

5. A face shield assembly according to claim 3, wherein the channel section is resiliently deformable and wherein the spacer element is provided by a web section and connects the inner flexible flange portion and outer mounting portion.

6. A face shield assembly according to claim 2, wherein the outer mounting portion comprises an outer flexible flange portion.

7. A face shield assembly according to claim 6, wherein the inner flexible flange portion is movable relative to the outer flexible flange portion.

8. A face shield assembly according to claim 6, wherein the outer flexible flange portion defines a constant spacing from the inner flexible flange portion.

9. A face shield assembly according to claim 6, wherein the inner flexible flange portion and/or outer flexible flange portion is substantially planar in vertical cross-section.

10. A face shield assembly according to claim 6, wherein the brow band comprises a respective tab extending from each end of the outer flexible flange portion, each tab having a means for attaching the face shield and/or elongate securing strap to the brow band.

11. A face shield assembly according to claim 10, wherein each tab comprises one or more cleats or projections for attaching the face guard and/or securing strap to the brow band.

12. A face shield assembly according to claim 10, wherein each tab comprises an elongate slot, the slot configured to receive an edge portion of the face guard to prevent rotation thereof relative to the brow band.

13. A face shield assembly according to claim 1, wherein the inner flexible flange portion is non-absorbent.

14. A face shield assembly according to claim 1, wherein the inner flexible flange portion and/or outer mounting portion is/are curved.

15. A face shield assembly according to claim 1, wherein the brow band is unitary.

16. A face shield assembly according to claim 1, wherein the brow band is formed of a plastics material and/or wherein the face guard is formed of a substantially transparent plastics material.

17. A face shield assembly according to claim 11, wherein the face guard comprises one or more apertures corresponding to the one or more cleats or projections, wherein the one or more apertures are configured to receive the projections so as to releasably attach the face guard to the brow band.

18. A face shield assembly according to claim 1 wherein at least part of the face guard is configured to be retained on the brow band between the brow band and securing strap.

19. A face shield assembly according to claim 1 wherein the securing strap is adjustable in length.

20. A face shield assembly according to claim 19, wherein the securing strap has a cable portion having a plurality of teeth and a releasable pawl configured to engage the teeth or wherein the securing strap has a stretchable portion and comprises a lattice structure formed of a strip of plastics material having a plurality of cute-outs.

21. (canceled)

22. (canceled)

23. A brow band for use in a face shield assembly, the brow band comprising:
an inner flexible flange portion configured to contact the brow of a wearer, an outer flexible flange portion comprising attachment means for releasable attachment of a face guard and a spacer element connecting the inner flexible flange portion and outer flexible flange portion.

24. A brow band according to claim 23, wherein the inner flexible flange portion, outer flexible flange portion and spacer element define a resiliently deformable channel section comprising a Z profile or U profile and having a respective tab extending from each end thereof, each tab having a means for releasably attaching a face guard and/or elongate securing strap to the brow band.
